(19) United States
(12) Patent Application Publication
(10) Pub. No.: US 2021/0121211 A1
(43) Pub. Date: Apr. 29, 2021

(54) Plating System For Repair of Femur Fractures

(71) Applicant: Joseph T. Labrum, IV, Philadelphia, PA (US)

(72) Inventor: Joseph T. Labrum, IV, Philadelphia, PA (US)

(73) Assignee: Mr. Joseph T. Labrum, IV, Nashville, TN (US)

(21) Appl. No.: 16/997,836

(22) Filed: Aug. 19, 2020

Related U.S. Application Data
(60) Provisional application No. 62/927,305, filed on Oct. 29, 2019.

(51) Int. Cl. A61B 17/72; A61B 17/80; A61B 17/74

(52) U.S. Cl. CPC A61B 17/7233; A61B 17/744; A61B 17/8061

(57) ABSTRACT

Current art lacks a plating system that allows for fixed angle plate and fastener placement that also provides adequate space for concurrent intramedullary nail placement and optionality of fastening the plate to the intramedullary nail with a fixed-angle construct for distal femur fracture fixation. The subject invention, which is a unique plating system for repairing distal femur fractures is described. The system includes a unique plate and a multitude of fasteners that are uniquely contoured to fit the distal femur and provide a fixed angle fixation system. The subject system has the optionality of application to the medial and/or lateral sides of the distal femur bone. Additionally, this plating system allows placement of a multitude of fasteners into the distal femur that can avoid violation of the femoral intramedullary canal. As a result, this system allows concurrent independent intramedullary rod fixation as well as optionality of fixed angle fastener placement through the plating system into an intramedullary rod for augmentation of fixation with a linked, fixed-angle plate-nail fixation construct.

### CROSS REFERENCE TO RELATED APPLICATIONS

Priority is claimed from provisional patent application U.S. Ser. No. 62/927,305 filed on Oct. 29, 2019 and incorporated by referenced herein.

### FIELD OF THE INVENTION

This invention relates to the field of fixation systems that are surgically implanted in the body of a patient for use in open reduction and internal fixation of bony fractures and/or non-unions. More specifically, this invention is uniquely configured for use in open repair via operative fixation of distal fractures of the native femur bone with or without the presence of intra-articular fracture extension into the knee joint. Furthermore, this field generally utilizes a specifically contoured bone plate(s) that fit to a corresponding and specific portion(s) of the lower extremity of the femur.

### ABSTRACT AND SUMMARY OF THE INVENTION

The subject invention, which is a unique plating system for repairing distal femur fractures with or without intra-articular fracture extension, is described. The system includes a unique plate and a multitude of fasteners that are uniquely contoured to fit the distal femur and provide a fixed angle fixation system. The subject system has the optionality of application to the medial and/or lateral sides of the distal femur bone that avoids unnecessary trauma to ligaments and soft tissue structures surrounding the knee. Additionally, this plating system allows placement of a multitude of fasteners into the distal femur that can avoid violation of the femoral intramedullary canal. As a result, this fixation system has the additional optionality of being used independently or in conjunction with another concurrently placed orthopaedic fixation device, known as an intramedullary nail/rod, to provide improved stability and fixation of a distal femur fracture. The proposed invention has a uniquely shaped plate configuration with a linear section, an end section angled to the linear section, and a central section therebetween with a void superiorly which can allow the unique ability to (1) place fasteners, including intramedullary rod fasteners and/or bony screw fasteners, into the distal femur in the proximal, central void above the plate and/or (2) interlock the fixation system into an intramedullary nail member via fixed angle fasteners placed through the middle section and/or central section of the plate into the intramedullary nail and through the distal femur bone. This system allows optionality for fixed angle fasteners to be placed through the plate into an intramedullary rod for augmentation of intramedullary rod fixation with a supporting fixed angle construct. Lastly, the unique contour and shape of this plate allows preservation of medial and lateral femoral collateral ligaments during placement. A method of repairing distal femur fractures with or without intra-articular extension with this plating system is described. Following provisional fracture reduction, the plating system is applied to the distal femur medially and/or laterally, yielding a fixed angle fixation construct. Additionally, this system can be used in cases where intramedullary rod fixation will be employed. Following intramedullary rod placement, this plating system is applied for fixed angle fixation of the distal femur fracture. This plating system can then be further augmented with fixed angle fastening of the plating system through the intramedullary rod, creating a linked plate-intramedullary fixation device.

### BACKGROUND

The femur is the longest bone in the body, beginning at the hip joint and extending to the knee. A femur fracture is typically sustained from high-energy impact and usually requires surgical intervention. Standard of care for a fracture in the diaphyseal shaft, or middle portion, of the femur entails fixation with an intramedullary nail (rod) that is placed within the inner canal (medullary canal) of the femur bone. Intramedullary rod fixation can be introduced into the medullary canal of the femur from the hip (antegrade placement) or from the knee joint (retrograde placement). Distal femur fractures are a common injury encountered in patients undergoing physical trauma and are difficult to fix. When fractures sustained in the femur are very distal, meaning near the articular surface of the knee joint, or intra-articular, meaning the fracture propogates into the articular surface of the knee joint itself, fixation with an intramedullary nail becomes extremely difficult or impossible. In these instances, distal femur fractures often require fixation with a large plate and screws. Antegrade and retrograde femoral intramedullary nail placement has distinct advantages over plate fixation for the treatment of femur fractures. Patients treated with an intramedullary nail are often able to bear weight on their injured extremity immediately after surgery whereas patients treated with a large plate are required to remain non-weightbearing for a prolonged period of time following surgery. Additionally, patients treated with intramedullary rod fixation have higher satisfaction scores post-operatively. Unfortunately, intramedullary rod fixation is often not employed in very distal femur fractures or distal femur fractures with intra-articular extension as there is very limited bone distally to fasten to the intramedullary rod. Additionally, current state of art plate fixation constructs for repairing distal femur fractures preclude the placement of an intramedullary device secondary to a plurality of fasteners that exist within the intramedullary canal where an intramedullary rod would be placed.

U.S. Pat. No. 9,968,389 B2, the disclosure of which is fully incorporated herein by reference, describes a fixation system for mending distal femur periprosthetic fractures, or fractures around artificial joints known as total knee replacements, with the use of plates, fasteners, and an intramedullary component placed in retrograde fashion. This system cannot be utilized in native distal femur fractures. Therefore, this system cannot be used in any femur fracture in which the patient has not undergone a previous total knee replacement. Additionally, this system cannot be used in conjunction with current state of art antegrade or retrograde intramedullary rods used in the treatment of femur fractures. Additionally, this system cannot be used in the absence of an intramedullary component, which must be placed in retrograde fashion through a total knee replacement. The footprint of this plate precludes the placement of additional intramedullary rod fasteners above the plate. Lastly, this system requires bilateral distal femur plating.

United States Patent publication number US 2015/05831 A1, the disclosure of which is fully incorporated herein by reference, describes a fixation system for mending distal femur fractures with a medially placed distal femur plate with fixed angle fasteners. This system does not have the option of placing fasteners through the plate while avoiding hardware placement within the intramedullary canal of the femur. This system also cannot be utilized adjacent to an intramedullary fixation device. Additionally, this plating system cannot be fastened to an intramedullary fixation device via linkage with fixed angle fasteners. This system also does not provide adequate space for intramedullary nail fastener placement proximal to the distal aspect of the plate secondary to the plate footprint design. Lastly, this system cannot be utilized on the lateral aspect of the distal femur.

There is currently no plating system that allows for fixed angle plate and fastener placement that also provides adequate space for intramedullary nail placement and fixation of the intramedullary nail fasteners proximal to the plate. Additionally, there is no current art that provides the option of fastening the plate to the intramedullary nail with a fixed-angle construct for distal femur fixation. This is biomechanically favorable when compared to standard intramedullary nail fixation with non-locking fasteners (“bolts” or “interlocks”). Non-locking intramedullary rod fasteners fail in a sequential manner (non-locking fasteners loosen one by one and introduce increasing instability to the fixation construct) while fixed-angle (“locking”) fastener fixation fails in tandem (all locking fasteners must fail at the same time for fixation construct to loosen). As a result, each additional locking fastener introduces additive biomechanical strength to the fixation construct. As the proposed plating system provides fixed-angle fixation and allows for additional fixed-angle fastener placement into the femur fracture fragments, it is biomechanically advantageous. Lastly, there is no plating system available that allows fixed angle fixation with or without concurrent intramedullary nail placement that can be placed on the medial or lateral native distal femur while allowing preservation of the medial and/or lateral collateral ligaments. Therefore, there is a clear need in the field of orthopaedics for an implant that provides these unique characteristics.

### BRIEF DESCRIPTION OF THE FIGURES

FIG. 1A—Anteroposterior view of the plating system without intramedullary nail augment

FIG. 1B—Lateral view of the plating system without intramedullary nail augment

FIG. 2A—Anteroposterior view of the plating system without intramedullary nail augment in an installed condition with intramedullary rod in place (dotted line) and fastener applied through plating system into intramedullary rod with additional intramedullary rod fasteners in place in central void of plate (some fasteners not shown)

FIG. 2B—Lateral view of the plating system without intramedullary nail augment in an installed condition with intramedullary rod in place (dotted line) and fastener applied through plating system into intramedullary rod with additional intramedullary rod fasteners in place in central void of plate (some fasteners not shown)

FIG. 3A—Anteroposterior view of the plating system with intramedullary nail augment

FIG. 3B—Lateral view of the plating system with intramedullary nail augment

FIG. 4A—Anteroposterior view of the plating system with intramedullary nail augment in an installed condition with intramedullary rod in place (dotted line) and a plurality of fasteners applied through plating system into intramedullary rod (some fasteners not shown)

FIG. 4B—Lateral view of the plating system with intramedullary nail augment in an installed condition with intramedullary rod in place (dotted line) and a plurality of fasteners applied through plating system into intramedullary rod (some fasteners not shown)

FIG. 5A—One potential depiction of the proposed plating system without intramedullary nail augment

FIG. 5B—One potential depiction of the proposed plating system with intramedullary nail augment

### DETAILED DESCRIPTION OF THE PREFERRED EMBODIMENT(S)

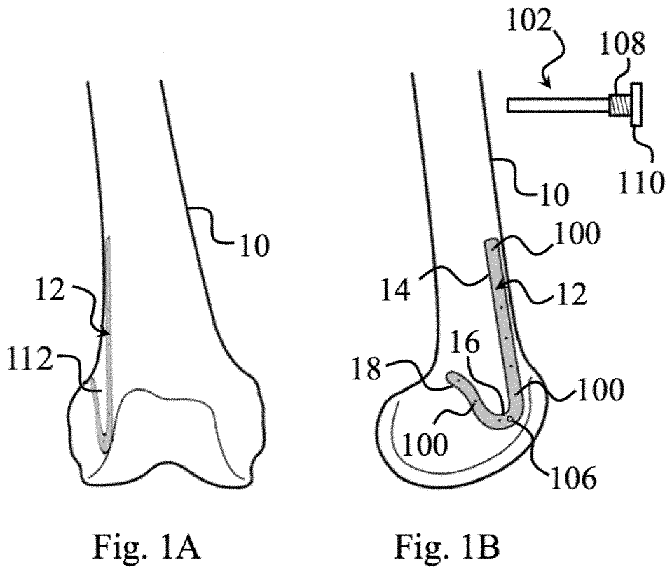

Shown in FIGS. 1A-5B, the subject system is presented in two modifications. The first modification or embodiment of the subject system is detailed in FIGS. 1A-1B, 2A-2B and 5A, and includes a plate 12 which has a linear portion 14, an end section 18, and a middle portion 16 which connects the linear portion 14 and the end section 18 of the plate 12, all of which are formed in an integral manner in one piece formation. In this modification, the plate 12 is provided with a plurality of openings (holes) 100 dispersed along the length of the plate. The openings 100 serve for insertion and securing of the fasteners 102 schematically presented in FIG. 1B, as will be detailed in further paragraphs.

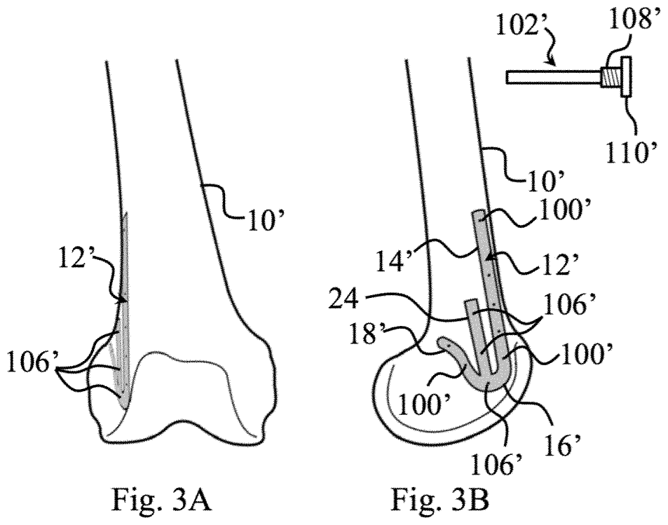

In another embodiment implementation, shown in FIGS. 3A-3B, 4A-4B, and 5B, the subject plating system includes a plate 12′, which is shaped with the linear portion 14′, end section 18′, and a middle portion 16′, connecting the linear portion 14′ and the end section 18′, all of which are formed integrally and in one piece formation. The plate 12′ is also configured with a central interlocking augment, or central section, 24 which is formed at the middle portion 16′ and extends therefrom between the linear portion 14′ and the end section 18′ of the plate 12′. The plate 12′ is configured with a plurality of openings 100′ which are dispersed one from another along the linear portion 14′, middle portion 16′, and end section 18′, as well as along the central section 24.

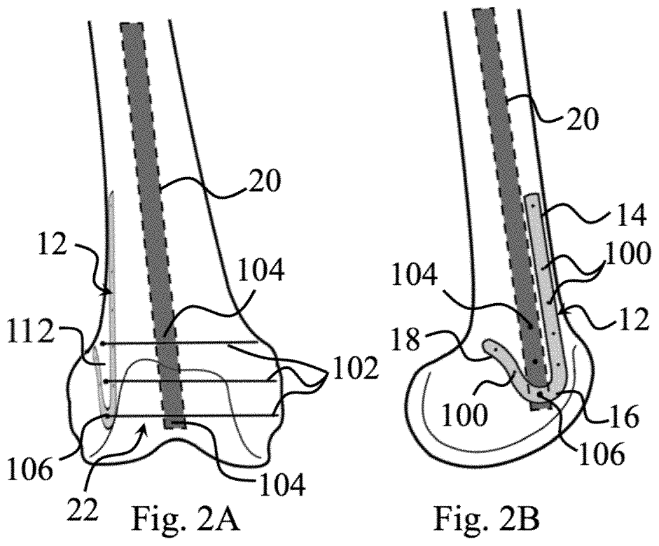

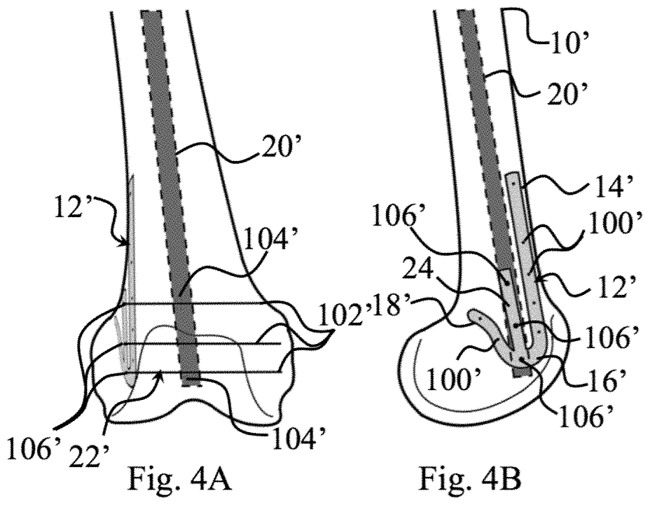

In operative application, either in the configuration of the plate 12 or in the configuration of the plate 12′, the plating system is implanted through a cut in the soft tissues overlaying the femur bone 10 and is compressed against the bone 10. The IM nail (rod) 20 shown in FIGS. 2A-2B, or the IM nail (rod) 20′ shown in FIGS. 4A-4B can be concomitantly inserted into the medullary cavity (canal) of the femur bone 10, through the knee joint or through the proximal aspect of the femur near the hip. The IM nail 20, 20′ assists in fixing the fragments of the femur bone separated by fracture. The intramedullary nail 20, 20′ is formed with a plurality of transverse openings 104 shown in FIGS. 2A, 2B, or openings 104′, shown in FIGS. 4A-4B.

The uniquely shaped plate 12, as shown in FIGS. 1A-1B and 2A-2B and 5A, and the uniquely shaped plate 12′ which is shown in FIGS. 3A-3B, 4A-4B, and 5B, have a special contour which is a somewhat J-shaped contour with the middle portion 16, 16′ connecting the linear portion 14, 14′ and the end section 18, 18′, respectively. This contour or shape provides additional points of fixation where an interlock fastener system 22 is placed through the plate 12, 12′ and the nail 20, 20′, respectively. The interlock fastener system 22 is formed with the fasteners 102, such as 3-5 mm locking fasteners, which pass through the linear portion 14, 14′, the end section 18, 18′, as well as the middle section 16, 16′, into the femur bone 10 in order to create a fixed-angle mechanism that holds fragments of the bone 10 at both sides of the fracture in place. The shape of the plate 12, 12′ provides a superior fixation of the fracture and an enhanced interlocking option between the plate 12, 12′ with the IM nail 20, 20′.

The end section 18, 18′ extends in a particular manner angled to the linear portion 14, 14′ of the plate 12, 12′, respectively, in order to match the configuration of the bone at the distal portion of the femur bone 10. In terms of fracture patterns, the unique configuration of the plate 12, 12′ may be of aid in positioning fasteners closer to the distal femur fracture and to follow the curve of the distal femur bone.

As shown in FIGS. 1A-1B, 2A-2B, 3A-3B, and 4A-4B, the configuration of the plate 12, 12′ is uniquely suited for repair of the fractures at the distal femur bone, where the medial or lateral ligaments are attached. It is necessary during surgical application to maintain medial or lateral ligamentous integrity, and the uniquely shaped plate 12,12′ allows application as well as plate and fastener placement without traumatic dissection of these ligamentous structures.

The central section 24 shown in FIGS. 3A-3B, 4A-4B, and 5B, is provided in the plate 12′ between the linear portion 14′ and the end section 18′ and extends from the middle portion 16′ in integral connection therewith (at one end of the central nail interlocking augment portion 24). Such construction provides additional points of fixation into the nail 20′. A surgeon performing the operation thus is equipped with the option to lock the J-configured plate 12′ to the IM nail 20′ in multiple places which increases mechanical support for the fractured bone.

The fastening system 22, 22′ includes a number of locking (fixed-angle) fasteners (screws) 102, 102′, for example, 3-5 mm locking screws or pegs, which slide through the openings 106, 106′ in the plates 12, 12′, through the bone 10, and through the openings 104, 104′ formed in the concurrently applied intramedullary nail 20, 20′.

The transverse openings (holes) 104 and 104′ in the intramedullary nail 20, 20′, respectively, extend through the IM nail 20, 20′ with the option to accommodate one or more fasteners 102 or 102′. Inserting one or more of the fasteners 102 or 102′ through the plate fixation device through the distal femur bone 10 can create a fixed angle device with absolute stability, preventing rotation and/or displacement of the bone fragments relative to one another about the axial, coronal, and sagittal axes of the intramedullary nail 20, 20′. The fasteners 102, 102′ may also be placed through the plate fixation system holes 106,106′ into the intramedullary nail 20, 20′ interlocking holes 104,104′, thereby locking the distal articular block/plate construct to the load sharing intramedullary device.

Preferably, each fastener 102, 102′ has a shaft, with or without a locking mechanism 108,108′ located under the head 110,110′ of the fastener, which lock into the plate 12,12′. The fasteners 102, 102′ which are used for the middle portion 16, 16′ of the plate 12, 12′ and the linear central nail interlocking augment 24 can be approximately 4 mm-5 mm thick locking screws or pegs, while the peripheral fasteners used for the linear portion 14, 14′ and the end section 18, 18′ of the plate 12, 12′ may be more variable in size, such as 3 mm locking screws or pegs.

The materials for the plate 12, 12′, as well as the fasteners 102, 102′ would be a strong material, such as 316L stainless steel. Alternatively, these structures may be formed from titanium, similar metallic compositions, carbon fiber, or other biomechanically favorable materials.

During the surgical operation, the plate 12, 12′ is compressed against the bone 10 which may be achieved by inserting each fastener 102, 102′ through a corresponding opening 100, 100′, respectively, in the plate 12, 12′. Additionally, at time of surgery if an intramedullary nail is placed, the plate 12,12′ can be interlocked into the intramedullary nail 20,20′ by placing fasteners through the plate holes 106, 106′ and subsequently into the intramedullary nail 20, 20′ through the intramedullary nail interlocking holes 104,104′.

It is preferred that the spacing between the openings 104, 104′ formed in the intramedullary nail (or rod) 20, 20′ is about the same as the spacing between the openings 106, 106′ formed in the plate 12, 12′, and can be aligned one with another for the fasteners passage therethrough.

The plates 12, 12′ are constructed to be attached to the bone 10 as close to the distal end thereof as possible, so that extreme distal and/or intra-articular fractures in the femur bone may be repaired. To enable attachment close to the distal end of the bone 10, the plate 12, 12′ is contoured to match the contour of the distal femur to which it is attached. When attached to the distal portion of the femur bone, the linear portion 14 of the plate 12 extends along the shaft of the femur bone while the middle portion 16 and the end section 18, which are the bent sections, may be applied around and over the distal portion of either the medial or lateral condyle of the femur.

With respect to the plate 12′, the linear portion 14′ of the plate 12′ extends along the shaft of the femur bone 10, while the middle portion 16′, end section 18′, and the central nail augment 24 are applied around and over at least a portion of either the medial or lateral condyle of the femur 10.

The subject unique plating system is designed for repairing distal femur fractures with or without intra-articular fracture extension in order to allow healing of these fractures. While the subject fixation system is displayed in FIGS. 1A-1B, 2A-2B, 3A-3B, 4A-4B, and 5A-5B, and described in the specification, the invented surgical system is not intended to be limited to the details shown and described in the current application.

The use of the subject fixation system is described herein (1) with the fixation system alone; (2) with the fixation system in the setting of concomitant intramedullary nail fixation; or, (3) with the fixation system fastened to concomitant intramedullary nail fixation.

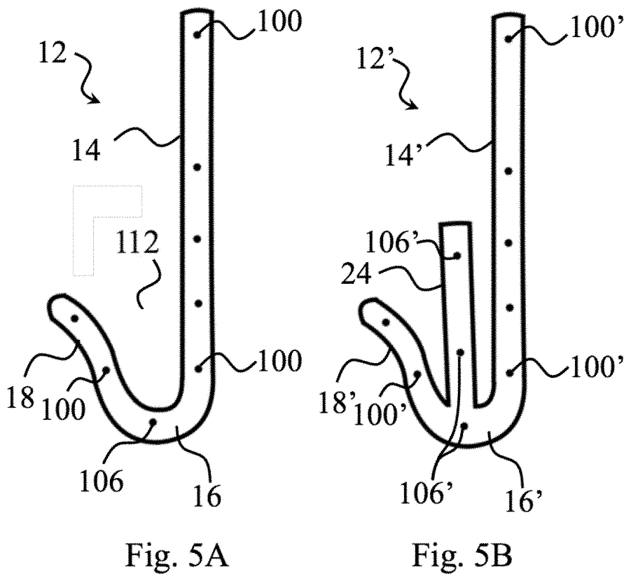

(1) Fixation system alone: Following fracture reduction and provisional fixation, the subject plating system 12, 12′ shown in FIGS. 5A-5B, respectively, is applied to the distal femur as shown in FIGS. 1A-1B, 3A-3B; and fasteners 102, 102′ are placed through the plate 12, 12′ into the broken bone 10, resulting in a fixed angle fixation construct.

(2) Concomitant intramedullary nail fixation: Following fracture reduction and provisional fixation, the intramedullary (IM) nail 20, 20′ start point is obtained. Following intramedullary nail start point attainment, the plate 12, 12′ is applied to the distal femur and fasteners 102, 102′ are placed through the plate 12, 12′ into the broken bone for a fixed angle fixation of the distal femur fracture. The intramedullary nail 20, 20′ is then placed into the medullary canal of the femur 10 and the transversely oriented fasteners 102, 102′ can be placed in the proximal central void 112 of the plating system.

(3) Fixation system fastened to concomitant intramedullary nail fixation: Following fracture reduction and provisional fixation, the intramedullary nail 20, 20′ start point is obtained. Following the intramedullary nail start point attainment, the plating system 12, 12′, with or without the central section 24, is applied to the distal femur. The intramedullary nail 20, 20′ is then placed into the medullary canal of the femur 10. The transversely oriented fastener openings 104, 104′ within the intramedullary nail 20, 20′, are then aligned with a similarly oriented fastener opening(s) 106, 106′ on the intramedullary nail plating system 12, 12′, with or without the central section 24. The fastening system 22, 22′ is subsequently realized with the fasteners 102, 102′ through the fixation system fastener opening(s) 106, 106′ as well as through the intramedullary nail fastener opening(s) 104, 104′. The remainder of the fixation system fasteners can then be placed through the system into the femur. The remainder of the intramedullary nail transversely oriented fasteners can be placed into the femur in the proximal central void 112 of the plating system 12 achieving interlock fastener system 22, or though the central section 24 on the plating system 12′ achieving interlock fastener system 22′, which has multiple points of fixed angle fixation to link the intramedullary nail and plating system.

Accordingly, other implementations are within the scope of the following claims. Changes may be made in the combinations, operations, arrangements, and depictions of the various parts and elements described herein without departing from the spirit and scope of the invention.

### REFERENCES CITED

### U.S. Patent Documents

| | | |
|---|---|---|
| US 62/927,305 | October 2019 | Labrum |
| U.S. Pat. No. 9,968,389 B2 | May 2018 | Garino |
| US 2015/05831 A1 | January 2015 | Sands |

### OTHER PUBLICATIONS

Hake et al., “Modern Implant Options for the Treatment of Distal Femur Fractures,” J Am Acad Orthop Surg, March 2019, doi: 10.5435/JAAOS-D-17-00706.
Langford et al., “Nailing of Proximal and Distal Fractures of the Femur: Limitations and Techniques,” J Orthop Trauma, June 2009, vol. 23, No. 5, pp. S22-S25.

I claim:

1. A plating system for repairing distal femur fractures comprising:
a unique plate with a linear portion, an end section, and a middle portion with variable length central section that is designed to fit the distal femur on the lateral and/or medial side while preserving collateral ligaments and soft tissues.
a multitude of fasteners that are designed to fit the distal femur and provide a fixed angle fixation system through the use of locking and non-locking fasteners placed into the plating system and through the distal femur bone.
a unique plate design and fastener orientation that allows optional fixation of distal femur fractures with a multitude of fasteners that can be applied in an orientation which avoids hardware placement within the femoral intramedullary canal.

2. A plating system for repairing distal femur fractures which, as a result of a unique plate design and fastener orientation, can be applied independently or in conjunction with an antegrade or retrograde intramedullary femoral nail.

3. A plating system for repairing distal femur fractures with a central section and/or middle section of the plate that allows optionality to interlock the fixation system into an intramedullary nail member via fixed angle fasteners placed through the plating system into the intramedullary nail and through the distal femur bone, thus creating a linked plating-intramedullary fixation device.

4. The bone plating system of claims 1-3 wherein said plating system is uniquely contoured in order to allow application to the medial and/or lateral aspect of the distal femur.

5. The bone plating system of claims 1-3 wherein said plating system is uniquely contoured to avoid removal and/or injury to collateral ligaments and soft tissue structures around the knee.

6. The bone plating system of claims 1-3 wherein said plating system design allows fixation of distal femur fractures with a multitude of fasteners that can be applied in an orientation which avoid hardware placement within the femur intramedullary canal, thus providing space for additional intramedullary rod fixation.

7. The bone plating system of claims 1-3 wherein said plating system design has a proximal, central void which allows placement of intramedullary rod fasteners and/or bony screw fasteners, into the distal femur in the proximal, central void of the plate, thus providing space for additional intramedullary fastener fixation at the discretion of the treating surgeon.

8. The bone plating system of claims 1-3 wherein said the plating system has a uniquely shaped plate configuration with a linear section, an end section angled to the linear section, and a middle section therebetween which can allow the unique ability to interlock the fixation system into an intramedullary nail member via fixed angle fastener placement through the middle section of the plate into the intramedullary nail and through the distal femur bone.

9. The bone plating system of claims 1-3 wherein said plating system has a uniquely shaped plate configuration with a linear section, an end section angled to the linear section, a middle section therebetween, and a central section which can allow the unique ability to interlock the fixation system into an intramedullary nail member at multiple locations via fixed angle fasteners placed through the middle section and/or central section of the plate into the intramedullary nail and through the distal femur bone.

10. The bone plating system of claims 1-3 wherein said plating system has a uniquely shaped plate configuration with a linear section, an end section angled to the linear section, a middle section therebetween, and a central section with various connections between the central section and the adjacent linear and end sections which can introduce variable plating system rigidity and allow the unique ability to interlock the fixation system into an intramedullary nail member via fixed angle fasteners placed through the end section and/or central section of the plate into the intramedullary nail and through the distal femur bone.

11. The bone plating system of claims 1-3 wherein said bone plating system is made of titanium.

12. The bone plating system of claims 1-3 wherein said bone plating system is made of stainless steel.

13. The bone plating system of claims 1-3 wherein said bone plating system is made of carbon fiber.

14. The bone plating system of claims 1-3 wherein said bone plating system is made of similar metallic compositions or other biomechanically favorable materials.

15. The bone plating system of claims 1-3 wherein said plating system has a uniquely shaped plate configuration with a central section that is less than 5 mm long.

16. The bone plating system of claims 1-3 wherein said plating system has a uniquely shaped plate configuration with a central section that is approximately 5-15 mm long.

17. The bone plating system of claims 1-3 wherein said plating system has a uniquely shaped plate configuration with a central section that is approximately 15-30 mm long.

18. The bone plating system of claims 1-3 wherein said plating system has a uniquely shaped plate configuration with a central section that is approximately 30-45 mm long.

19. The bone plating system of claims 1-3 wherein said plating system has a uniquely shaped plate configuration with a central section that is approximately 45-60 mm long.

20. The bone plating system of claims 1-3 wherein said plating system has a uniquely shaped plate configuration with a central section that is greater than 60 mm long.

21. The bone plating system of claims 1-3 wherein top surface of said bone plating system is at a distance of less than 5 millimeters to bottom surface that directly contacts bone.

22. The bone plating system of claims 1-3 wherein top surface of said bone plating system is at a distance of approximately 5 millimeters to bottom surface that directly contacts bone.

23. The bone plating system of claims 1-3 wherein top surface of said bone plating system is at a distance of greater than 5 millimeters to bottom surface that directly contacts bone.

24. The bone plating system of claims 1-3 wherein said apertures designed to fit fasteners in the central section and/or middle section that can interdigitate with an intramedullary rod at a specific distance from one another to allow coaxial alignment with intramedullary rod fastener apertures spaced by 0-5 mm.

25. The bone plating system of claims 1-3 wherein said apertures designed to fit fasteners in the central section and/or middle section that can interdigitate with an intramedullary rod at a specific distance from one another to allow coaxial alignment with intramedullary rod fastener apertures spaced by 5-10 mm.

26. The bone plating system of claims 1-3 wherein said apertures designed to fit fasteners in the central section and/or middle section that can interdigitate with an intramedullary rod at a specific distance from one another to allow coaxial alignment with intramedullary rod fastener apertures spaced by 10-15 mm.

27. The bone plating system of claims 1-3 wherein said apertures designed to fit fasteners in the central section and/or middle section that can interdigitate with an intramedullary rod at a specific distance from one another to allow coaxial alignment with intramedullary rod fastener apertures spaced by 15-20 mm.

28. The bone plating system of claims 1-3 wherein said apertures designed to fit fasteners in the central section and/or middle section that can interdigitate with an intramedullary rod at a specific distance from one another to allow coaxial alignment with intramedullary rod fastener apertures spaced by 20-25 mm.

29. The bone plating system of claims 1-3 wherein said apertures designed to fit fasteners in the central section and/or middle section that can interdigitate with an intramedullary rod at a specific distance from one another to allow coaxial alignment with intramedullary rod fastener apertures spaced by 25-30 mm.

30. The bone plating system of claims 1-3 wherein said apertures designed to fit fasteners in the central section and/or middle section that can interdigitate with an intramedullary rod at a specific distance from one another to allow coaxial alignment with intramedullary rod fastener apertures spaced by more than 30 mm.

31. The bone plating system of claims 1-3 wherein said end section is angled to the linear section by less than 10 degrees.

32. The bone plating system of claims 1-3 wherein said end section is angled to the linear section by approximately 10-15 degrees.

33. The bone plating system of claims 1-3 wherein said end section is angled to the linear section by approximately 15-30 degrees.

34. The bone plating system of claims 1-3 wherein said end section is angled to the linear section by approximately 30-45 degrees.

35. The bone plating system of claims 1-3 wherein said end section is angled to the linear section by greater than 45 degrees.
